(19) United States
(12) Plant Patent Application Publication
(10) Pub. No.: US 2017/0367231 P1
(43) Pub. Date: Dec. 21, 2017

(54) Apple Tree Named 'Venice'

(50) ;

(71) Applicant: Empresa de Pesquisa Agropecuária e Extensão Rural de Santa Catarina (Epagri), Florianopolis (BR)

(72) Inventors: Frederico Denardi, Cacador (BR); Marcus Vinicius Kvitschal, Cacador (BR); Maraisa Crestani Hawerroth, Cacador (BR)

(73) Assignee: Empresa de Pesquisa Agropecuária e Extensão Rural de Santa Catarina (Epagri), Florianopolis (BR)

(21) Appl. No.: 14/999,707

(22) Filed: Jun. 15, 2016

(51) Int. Cl. A01H 5/08

(52) U.S. Cl. CPC A01H 5/0875
USPC PLT/161

(57) ABSTRACT

‘Venice’ apple tree (Malus domestica Borkh) is a new variety selected for its low chilling requirement, resistance to Gala leaf spot disease and partial horizontal resistance to apple scab, high fruit quality, and excellent storageability.

Latin name: Malus domestica Borkh.

Variety denomination: ‘Venice.

### BACKGROUND OF THE VARIETY

‘Venice’ is a new and distinct variety of apple tree (Malus domestica Borkh) obtained from a controlled cross of ‘Imperatriz’ (female parent, not patented)×‘Baronesa’ (male parent, not patented) carried out at Caçador, Santa Catarina, Brazil in 2000. Seeds obtained from the cross were planted at Caçador, and ‘Venice’ was selected from the resulting seedlings for propagation and further observation. ‘Venice’ was first asexually propagated by grafting in 2004 at Caçador, and has since been observed to remain true to type over successive asexually propagated generations.

### BRIEF DESCRIPTION OF THE VARIETY

‘Venice ’ apple tree is distinguished by its resistance to Gala leaf spot disease (Colletotrichum spp.) and partial horizontal resistance to apple scab (Venturia inaequalis), and its low chilling requirement. Fruit of ‘Venice’ is notable for its high quality, having crisp juicy flesh and a high sugar content, and its excellent storageability.

‘Venice’ is distinguished from female parent ‘Imperatriz’ by its smaller, more acidic fruit; later ripening; more intense skin color; and improved storageability.

‘Venice’ is distinguished from male parent ‘Baronesa’ by its earlier fruit maturity (10 to 15 days earlier than ‘Baronesa’); more coarse flesh texture; higher acidity; and more pronounced overcolor.

‘Venice’ is distinguished from standard ‘Fuji’, a similar variety, by its lower chilling requirement which is particularly suited to growing conditions in Brazil; partial horizontal resistance to apple scab; lower tendency to biannual bearing; production of fruit predominantly on spur buds, as compared to ‘Fuji’, which produces fruit primarily on terminal buds of long shoots; more complete and intense overcolor on fruit; more coarse fruit texture; improved storageability; and increased susceptibility to penicilium fruit rot (Penicilium espancium) during storage.

### BRIEF DESCRIPTION OF THE PHOTOGRAPHS

The accompanying photographs of the ‘Venice’ variety were taken from 2013 to 2015 at Caçador, Brazil.

FIG. 1 is a photograph of a ‘Venice’ apple;

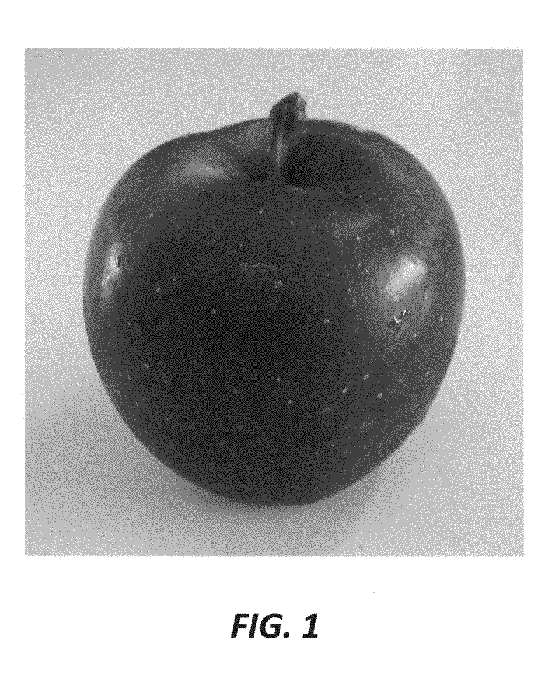

FIG. 2 is a photograph of ‘Venice’ apples on the tree;

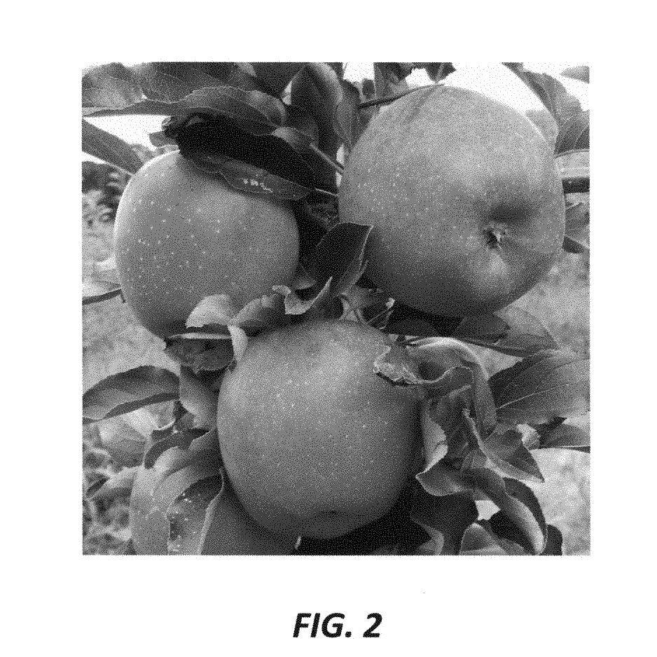

FIG. 3 is a photograph of ‘Venice’ apple trees in bloom; and,

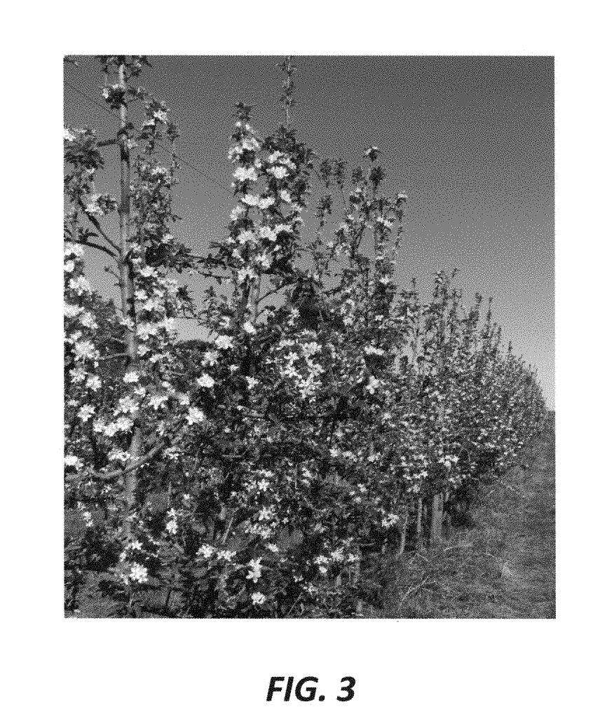

FIG. 4 is a photograph of ‘Venice’ apple trees and fruit.

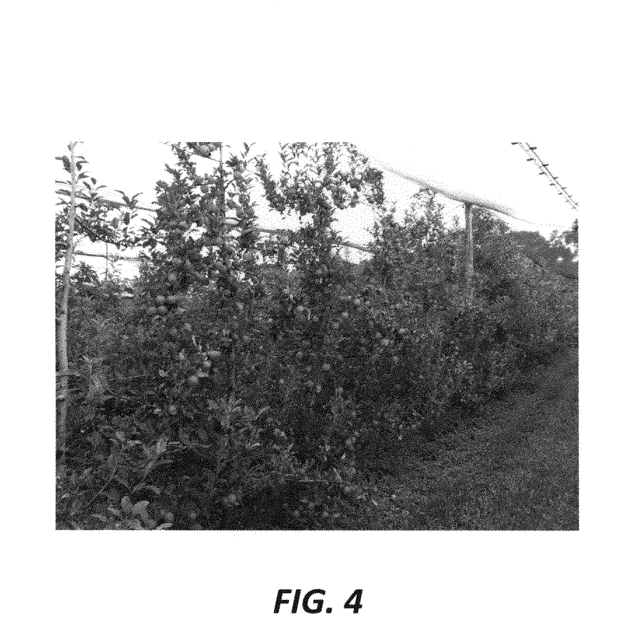

### DETAILED BOTANICAL DESCRIPTION OF THE VARIETY

The following-detailed botanical description is based on observations made at Caçador, Brazil from 2013 to 2015, of trees planted in 2011, grown on ‘Marubakaido’ rootstock (not patented) with ‘M-9’ interstock (not patented). All colors are described according to The Royal Horticultural Society Colour Chart. It should be understood that the characteristics described will vary somewhat depending upon cultural practices and climatic conditions, and will vary with location and season. Quantified measurements are expressed as an average of measurements taken from a number of individual plants of the new variety. The measurements of any individual plant or any group of plants of the new variety may vary from the stated average.
Tree:
   Vigor.—Medium.
   Type.—Ramified.
   Habit.—Spreading.
   Height.—3.6 m.
   Trunk diameter (at 30 cm above the graft).—8.1 cm.
   Bark texture.—Slightly rough.
   Bark color.—Yellow-green 148C.
   Lenticel size.—1.6 mm.
   Lenticel color.—Greyed-yellow 161A.
Branch (fruiting branches located at around 1 m above the graft union):
   Length.—1.1 m.
   Diameter.—26 mm.
   Crotch angle.—80° to 85°.
   Bark color.—Greyed-green 198A.
   Lenticel length.—1 mm.
   Lenticel color.—Yellow-white 158A.
   Number of lenticels per cm2.—3.2.
One year old shoot:
   Length.—0.7 m.
   Color.—Greyed-purple 187A.
   Pubescence.—Weak.
   Thickness.—6 mm.
   Internode length.—25 mm.
   Number of lenticels per cm2.—1.77.
Flower buds:
   Quantity per spur.—1.33.
   Shape.—Spiky.
   Length.—8 mm.
   Diameter.—4 mm.
   Color.—Greyed-purple 183A.
Flowers:
   Date of first bloom.—September 10 to September 18.
   Date of full bloom.—September 25 to October 3.
   Pollination requirement.—Yes.
Leaves:
   Shape.—Ovate.
   Length.—10 cm.
   Width.—5 cm.
   Length/width ratio.—2.
   Blade margin.—Crenate.
   Apex shape.—Acute.
   Base shape.—Rounded.
   Color of upper surface.—Green N137A.
   Color of lower surface.—Yellow-green 146D.
   Attitude in relation to shoot.—Upward.
Petiole:
   Length.—33 mm.
   Diameter.—2 mm.
   Color.—Yellow-green 148A.
Fruit:
   Diameter.—70 mm.
   Height.—64 mm.
   Weight.—173 g.
   Ratio of height to width.—1.09.
   General shape in profile.—Conical.
   Position of maximum diameter.—On the third near to the stalk cavity.
   Ribbing.—Narrow.
   Crowning at calyx end.—Weak.
   Size of eye.—6 mm.
   Aperture of eye.—7 mm.
   Length of sepal.—7 mm.
   Bloom of skin.—Absent to weak.
   Greasiness of skin.—Absent to weak.
   Background color of skin.—Green-yellow 1C.
   Over color of skin.—Orange-red N34A.
   Amount of over color.—70% to 90%.
   Intensity of over color.—Medium.
   Pattern of over color.—Solid flush with weakly defined stripes.
   Amount of russet around stalk cavity.—Medium.
   Amount of russet on cheeks.—Absent.
   Area of russet around eye basin.—Absent.
   Length of stalk.—19 mm.
   Thickness of stalk.—3 mm.
   Depth of stalk cavity.—8 mm.
   Width of stalk cavity.—26 mm.
   Depth of eye basin.—11 mm.
   Width of eye basin.—20 mm.
   Firmness of flesh.—18 to 18.5 pounds.
   Flesh texture.—Fine.
   Aroma.—Medium.
   Juiciness.—High.
   Brix.—14° to 14.5° Brix.
   Flesh color.—Yellow 4D.
   Stem color.—Greyed-brown N199D.
Seeds:
   Quantity per fruit.—7.8.
   Shape.—Ovate.
   Color.—Greyed-orange 166A.
Harvest:
   Harvest date (actual and relative).—Mid-season (second half of March in Brazil).
   Amount of fruit produced per tree per harvest.—20 to 25 kg per tree.
Disease resistance/susceptibility: Susceptible, but with some horizontal resistance to apple scab (Venturia inaequalis) — not immune; Resistant to Gala leaf spot (Colletotrichum spp.); tolerance to powdery mildew (Podosphaera leucotricha); Susceptible to penicilium fruit rots in storage (Penicilium espancium).
Market use: Fresh.

1. I claim a new and distinct apple tree substantially as described and illustrated herein.
